(19) United States
(12) Patent Application Publication
(10) Pub. No.: US 2023/0321298 A1
(43) Pub. Date: Oct. 12, 2023

(54) Room Sanitization System

(71) Applicants: Kim Rozier, Columbus, GA (US); Tom Scott, Columbus, GA (US); Tristan Rozier, Columbus, GA (US)

(72) Inventors: Kim Rozier, Columbus, GA (US); Tom Scott, Columbus, GA (US); Tristan Rozier, Columbus, GA (US)

(21) Appl. No.: 17/990,856

(22) Filed: Nov. 21, 2022

Related U.S. Application Data
(60) Provisional application No. 63/290,047, filed on Dec. 15, 2021.

(51) Int. Cl. A61L 2/22; A61L 2/10; B05B 5/16

(52) U.S. Cl. CPC A61L 2/22; A61L 2/10; B05B 5/1608; A61L 2101/20

(57) ABSTRACT

A disinfecting system for a room having at least a first and second wall defining a room dimension including a housing having a reservoir containing a disinfecting fluid, a nozzle assembly including a nozzle and a nozzle controller for dispensing the disinfecting fluid into a room, and a pump for communicating the disinfecting fluid from the reservoir to the nozzle assembly. A controller controls the dispensing of the disinfectant. A dispensing profile controls the dispensing of said disinfectant. The dispensing profile utilizes the dimensions of the room as instructions for the central controller to identify the range for dispensing the disinfecting fluid. The central controller utilizes the dispensing profile instructions for manipulating either the pump, fan or nozzle assembly to dispense a desired volume of the disinfectant fluid at a range as identified by the dispensing profile. Wherein the amount of sanitization fluid dispensed will remain in a fluid state within the room constituting a dwell time of at least between one minute and ten minutes.

### PRIORITY

This application claims priority to previously filed Provisional Application entitled ECO Breeze filed Dec. 15, 2021 having Ser. No. 63/290,047.

### TECHNICAL FIELD

The present invention is directed towards a room sanitization system in general and a room sanitization system in particular which utilizes an electrostatic fluid delivery system for sanitizing a room in a fast, and efficient manner by utilizing an automated delivery system.

### BACKGROUND OF THE INVENTION

With the onset of the coronavirus COVID-19, the awareness for the need of sanitized facilities has increased. Prior sanitization of infectious diseases in public places such as hospitals, schools, restaurants, hotels has been undertaken by various methods typically involving manually spraying a fluid disinfectant onto surfaces and wiping down the surfaces with a cloth. Unfortunately, such cleaning methods are ineffective, timely and costly. A drawback is with the time involved, certain rooms are not available for an extended period of time for occupation or utilization. Such delays are not conducive to a hospital environment where patient rooms and operating rooms are generally needed for immediate occupancy or utilization. Furthermore, the more time that an operating room is out of service, the fewer operations and procedures may be conducted which promotes scheduling delays and also loss business opportunities for the respective hospitals.

Additional methods of sanitizing consist of utilizing various sprayers carried by individuals and manually canvassing an area while spraying. Such systems are expensive and time consuming. Other methods include spraying an entire area with a sprinkler type system or a localized area. Such systems are not very effective as they rely on the strategy of over saturation to ensure covering an entire area. Such systems leave a large residue of the disinfectant rendering the area unusable until the area dries out. These systems are impractical for common public areas and also render the risk of leaving slippery residue on the floors rendering the area a hazard.

Additionally, sanitization of hospitals is an ongoing concern. Typically, a janitorial crew which is very labor intensive is tasked with cleaning up hospital rooms and other areas of the hospital which includes operating rooms. The individuals wipe down various areas within the rooms in the hopes of eliminating any hazardous germs and viruses. The wipe down typically includes areas which have been exposed to contact by patients or visitors and can leave untreated areas. General patient rooms seldom receive hard cleaning as the room is usually prepped for the arrival of the next patient. By leaving the cleaning for humans, errors in the cleaning exists and with the cleaning being laborious, the cleaners are typically rushed to do the job missing areas which require attention.

Alternatively, operating rooms typically receive a different sanitization cleaning utilizing UV device which resemble “R2D2” robots. These standalone devices are positioned within the operating room and operate for a certain period of time utilizing UV lighting. Such sanitization takes a long time and are usually deployed in the evening when all surgeries are done for the day. Consequently, after the first surgery is done the next day, the room is left to be cleaned by a janitorial crew with the same errors which are common with human oversight.

Consequently, the sanitization of hospitals is an ongoing concern. Hospitals rely on the skills of the janitorial team — which cannot always be counted on. Poor sanitization opens the hospitals for liabilities as patients may contract MSRAs and other infectious diseases which exist due to the nature of the individuals/patients which preceded the patient in the hospital room or operating room.

Also, the contaminants which are present may vary and require different cleaning processes to ensure that the particular contaminant is destroyed. These processes require different exposure or contact times for the disinfectant to engage the contaminant and human error arise preventing

Accordingly, there is a need to provide for a room sanitization system which is effective and rendered in a timely manner ensuring that the public area may be subsequently used as soon as possible.

Furthermore, there is a need to provide a room sanitization system which may be conducted automatically in a manner which ensures that the various contaminants are destroyed by ensuring that the dwell time of the disinfectant is sufficient.

Additionally, there is a need to provide for an effective hospital sanitization system which quickly and readily sanitizes a hospital room or operating room so a clean and sterile environment is present which enables a room to be turned over quickly for the next patient or operation.

### SUMMARY OF THE INVENTION

A disinfecting system for a room having at least a first and second wall defining a room dimension, comprising a housing and a reservoir containing a disinfecting fluid carried by the housing. A nozzle assembly including a nozzle and a nozzle controller for dispensing the disinfecting fluid into a room, the nozzle assembly carried by the housing. A pump for communicating the disinfecting fluid from the reservoir to the nozzle assembly. The pump carried by the housing. A controller for controlling the dispensing of the disinfectant. The controller carried by the housing. A dispensing profile for controlling the dispensing of the disinfectant. The dispensing profile utilizing the dimensions of the room as instructions for the central controller to identify the dispensing the disinfecting fluid. The central controller utilizing the dispensing profile instructions for manipulating either the pump, fan or nozzle assembly to dispense a desired volume of the disinfectant fluid at a range as identified by the dispensing profile. Wherein the amount of sanitization fluid dispensed will remain in a fluid state within the room constituting a dwell time of at least between one minute and ten minutes.

A disinfecting system for a first room having at least a first and second wall defining a first room dimension and a second room having at least a first and second wall defining a second room dimension. The system includes a first disinfecting unit carried within the first room comprising a housing and a reservoir containing a disinfecting fluid carried by the housing. A nozzle assembly including a nozzle and a nozzle controller for dispensing the disinfecting fluid into a room, the nozzle assembly carried by the housing. A pump for communicating the disinfecting fluid from the reservoir to the nozzle assembly, the pump carried by the housing. A controller for controlling the dispensing of the disinfectant. The controller carried by the housing. A first dispensing profile for controlling the dispensing of the disinfectant. The first dispensing profile utilizing the dimensions of the first room as instructions for the central controller to identify the range for dispensing the disinfecting fluid from the first disinfecting unit. The central controller utilizing the first dispensing profile instructions for manipulating either the pump, fan or nozzle assembly to dispense a desired volume of the disinfectant fluid at a range as identified by the first dispensing profile to provide the disinfecting fluid to a first sanitization area as defined by the first dispensing profile and wherein the amount of sanitization fluid dispensed will remain in a fluid state within the room constituting a dwell time of at least between one minute and ten minutes. Also, second disinfecting unit carried within the second room having a housing and a reservoir containing a disinfecting fluid carried by the housing. A nozzle assembly including a nozzle and a nozzle controller for dispensing the disinfecting fluid into a room, the nozzle assembly carried by the housing. A pump for communicating the disinfecting fluid from the reservoir to the nozzle assembly, the pump carried by the housing. A controller for controlling the dispensing of the disinfectant. The controller carried by the housing. A second dispensing profile for controlling the dispensing of the disinfectant. The second dispensing profile utilizing the dimensions of the second room as instructions for the central controller to identify the range for dispensing the disinfecting fluid from the second disinfecting unit. The central controller utilizing the second dispensing profile instructions for manipulating either the pump, fan or nozzle assembly to dispense a desired volume of the disinfectant fluid at a range as identified by the second dispensing profile to provide the disinfecting fluid to a second sanitization area as defined by the second dispensing profile. Wherein the amount of sanitization fluid dispensed will remain in a fluid state within the room constituting a dwell time of at least between one minute and ten minutes; and the first sanitization area being within the first room and the second sanitization area being within the second room.

### DESCRIPTION OF THE DRAWING FIGURES

The foregoing objects and advantages of the present invention may be more readily understood by one skilled in the art with reference being had to the following detailed description of a preferred embodiment thereof, taken in conjunction with the accompanying drawings wherein like elements are designated by identical reference numerals throughout the several views, and in which:

FIG. 1 is an illustration of a room sanitization system according to an embodiment of the present invention;

FIG. 2 is perspective view of an electrostatic fluid sanitization system according to an embodiment of the present invention with a straw disposed therein;

FIG. 3 illustrates the internal components of the electrostatic fluid sanitization systems according to an embodiment of a present invention;

FIG. 4 illustrates the central processing system which controls the operation of the components of the system according to an embodiment of the present invention;

FIG. 5 illustrates a network of sanitization systems integrated to facilitate the sanitization of the multi-room establishment according to an embodiment of the present invention;

FIG. 6 illustrates a UV light system for sterilizing the ambient air according to an embodiment of the present invention;

FIG. 7 illustrates the creation of a the dispersion profile according to an embodiment of the present invention;

FIG. 8 illustrates the dispersion profile for the room shown in FIG. 7 according to an embodiment of the present invention;

FIG. 9 illustrates an additional room configuration with a system of multiple sanitization systems according to an embodiment of the present invention;

FIGS. 10A, 10B, 10C illustrate the respective dispersion profiles for the multiple sanitization systems shown in FIG. 9;

FIG. 11 illustrates the implementation of the sanitization system in the hospital setting either for patient care rooms or operating rooms according to the present invention.

### DETAILED DESCRIPTION

Before the present subject matter is further described, it is to be understood that this subject matter described herein is not limited to particular embodiments described, as such may of course vary. It is also to be understood that the terminology used herein is for the purpose of describing a particular embodiment or embodiments only and is not intended to be limiting. Unless defined otherwise, all technical terms used herein have the same meaning as commonly understood by one skilled in the art to which this subject matter belongs.

Disclosed herein is an electrostatic fluid delivery system that is configured to deliver fluid, such as a disinfectant fluid, into the air and subsequently onto a surface by electrically charging the fluid and forming the fluid into a mist, fog, plume, or spray that can be directed into the air and onto a surface. The system atomizes the fluid using a high-pressure air (or other gas) stream and passes the fluid through an electrode inside a nozzle assembly to charge, such as negatively charge, droplets of the atomized fluid. The device can electrically charge a liquid or gas.

The system is configured to electrostatically charge the atomized fluid via direct charging, induction charging, indirect charging, or any combinations thereof In the case of direct charging, fluid flows through a charging system. For induction or indirect charging, the fluid is passed through a medium, such as air, which has been electrostatically charged by one or more electrodes or pins that create a static electric field through which the fluid passes to receive a charge. The electrode may or may not be in the fluid stream.

The room sanitization system utilizes an electrostatic delivery system that sprays an electrically charged fluid, such as a disinfectant, into the air which will utilize the charge of the walls and the ceiling for facilitating the dispersion of the sanitization fluid throughout the entire room and respective surfaces. In the electrostatic delivery system, the sanitization fluid is atomized by a high-pressure air stream as it passes through an electrode inside a nozzle or housing. Negatively charged particles are thereby induced onto droplet surfaces of the solution to form electric field charge within the spray plume of the solution which acts in association with the naturally charged surfaces of the wall, ceilings and furniture surfaces to facilitate in the dispersion of the sanitization fluid throughout the room.

The electrostatic charge causes the fluid to cling to a surface to increase the likelihood that the disinfectant will cover and clean the surface. Additionally, by being charged, the disinfectant is induced to spread along a surface due to the opposite charges attracting the disinfectant over the respective surfaces. In essence, the disinfectant creeps over the surface area to cover the surface area. The electrostatic charge also causes the fluid to cling to the walls and the ceilings which act to draw the electrostatically charged sanitization fluid along throughout the airspace adjacent the ceiling the walls. The synergistic effect of the electrostatically charged sanitization fluid and the respective structure is that the fluid is drawn throughout the respective adjacent airspace due to the charged particles being attracted to the non-charged air particles. This system enables the fluid to be dispersed throughout the volume of the air space and not just in a linear flow. An additional benefit is that the air volume within the room is also sanitized by interaction with the sanitization fluid.

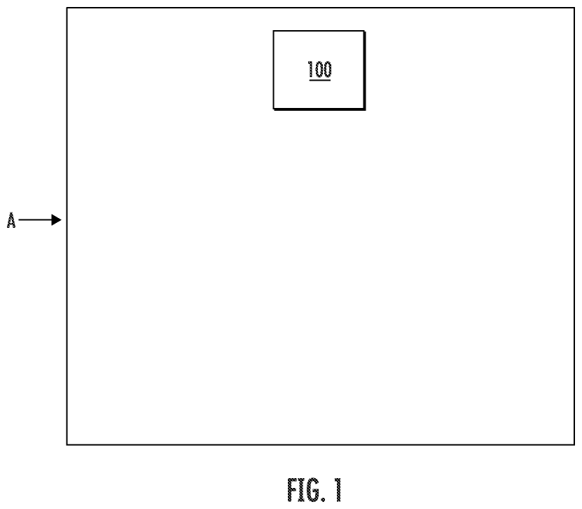

As shown in FIG. 1, the electrostatic fluid sanitization system 100 is designed for installation within the interior of room A. Preferably the system 100 is installed along a wall preferably two feet from the ceiling. Additional installation preferences will be discussed hereinafter depending on the overall configuration of the room. Such installation enables the charged sanitization material to be influenced along the walls and ceilings facilitating in the dispersion of the sanitization material throughout the entire volume of the room and not just specific surfaces. By focusing on directing the sanitization material throughout the entire air space volume, the many surfaces located within the room, along with the walls, ceilings and airspace itself are subject to the sanitization effects of the dispersed sanitization material.

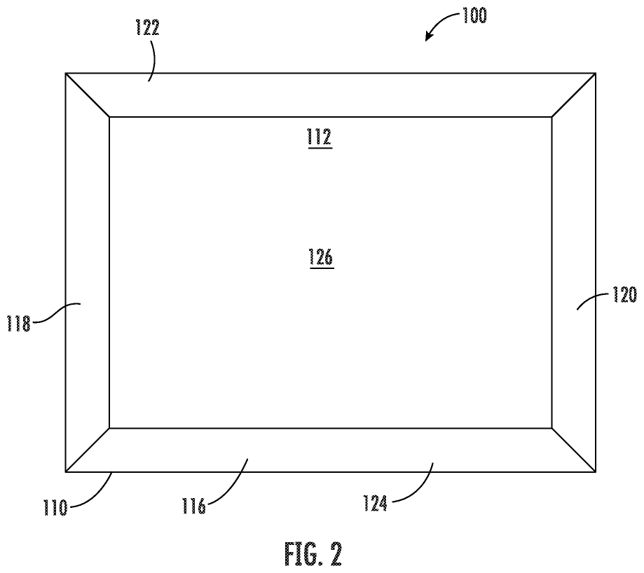

FIG. 2 shows a perspective view of an electrostatic fluid sanitization system 100 that is configured to electrically charge and atomize a fluid for spraying into the air in a room A which includes a ceiling, walls, and a floor. The electrostatic fluid sanitization system 100 includes a housing 110 which includes a primary body 112 having a back portion 114, a front portion 116, a left or first side 118, a right or second side 120, a top side 122 and a bottom side 124 forming a general box defining a housing interior 126 for receiving components of the electrostatic fluid sanitization system. To permit operation, various openings are defined within the housing components. These openings are utilized in certain circumstances as inlets 130 to permit air from the ambient environment to be drawn into the interior via a fan 132. Also, openings are utilized as dispensing outlets for facilitating the operation of nozzles for dispensing a sanitization fluid into the ambient environment.

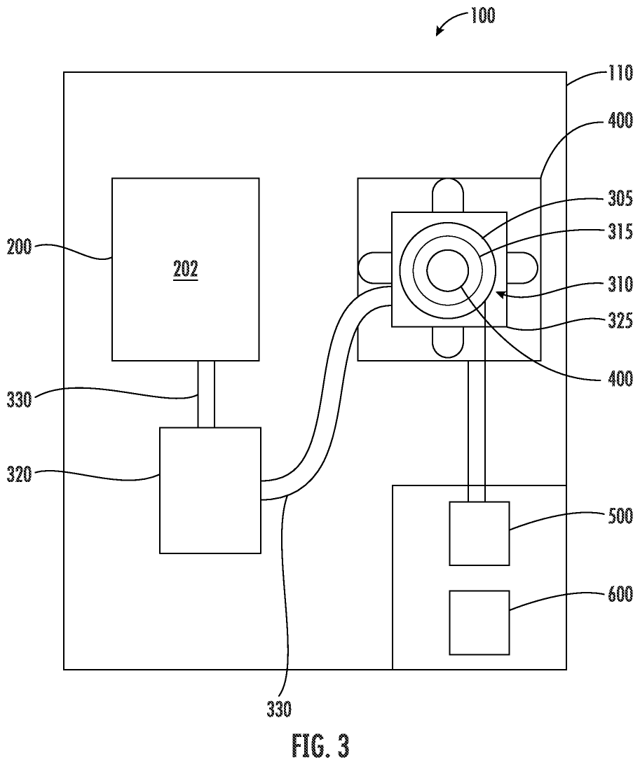

FIG. 3 illustrates the internal components of the electrostatic fluid sanitization system 100. A reservoir 200 contains a sanitizing fluid 202. Sanitizing fluid 202 is preferably a hypochlorous acid containing fluid. One such fluid is known as Envirocleanse-A having EPA Registration No. 85134-1. The Envirocleanse-A solution is for use on hard non-porous inanimate surfaces and is effective for disinfection of cabapenem resistant kiebsiella pneumoniae, gram positive and gram negative bacteria such as Staphylococcus aeureus and Escherichia coli. The solution is also effective against certain bacteria, viruses, fungi and SARS-Covid 2 as identified in Attachment A which has been provided to the EPA and incorporated by reference. Depending on the contaminant, certain dwell times or the interaction time of the sanitizing fluid with the respective contaminant will vary. These dwell times may vary from one minute to ten minutes. In addition to the type of contaminant, such as Covid-2, TB, bacteria, MSRA, the contaminant may be classified as a touch contaminant which is exchanged via a touch, airborne, which is spread throughout the air.

The sanitizing fluid contained within reservoir 200 is ultimately dispensed via a nozzle 315. Nozzle 315 atomizes and expels the sanitization fluid in a spray. Nozzle 315 is part of nozzle assembly 310. Nozzle assembly 310 includes an annular housing 305 having a central opening in which is positioned nozzle 315. The nozzle assembly 310 is mechanically coupled to a nozzle drive assembly 325 that moves the nozzle assembly 310 in a one hundred and seventy degree horizontal rotation and a one hundred and seventy degree vertical direction from a central location. In this manner, the user can move the nozzle in a wide range defining a spray profile for the dispersion of the sanitization fluid. Nozzle drive assembly 325 directs the movement of the nozzle assembly 310. The nozzle assembly 310 may be pivoted in an up and down motion to direct the sanitization spray and also in a left to right motion. The pivoting nature of the nozzle enables the nozzle to provide for a directed sanitization coverage which is desired based on the configuration of the particular room which the assembly is located.

The nozzle assembly 310 may include a charging element 400 which encircles the nozzle tips. The charging element 400 is electrically connected to the system and provides a voltage on the charging element such that as the nozzle 315 expels the atomized fluid through the charging element 400 the fluid is Negatively charged. The charging system is positioned along an axis that is in parallel to an axis of a spray nozzle so that the spray and ions are emitted in the same direction and along a parallel axis and therefore the droplets in the spray are surrounded and covered by ion stream and can be efficiently charged when they meet the ion stream. The electrodes thus emit, propel, or otherwise send out ions or charge in a direction parallel to the direct of fluid flow or an average direction of fluid flow from the nozzles. In this manner, the electrically charged plume of fluid will cling to surfaces that it is sprayed upon and also be attracted to the walls and ceilings, both in proximity and distal. Alternative charging systems may be deployed.

Any of a variety of nozzle types can be used to achieve a desired flow pattern. In the preferred embodiment, nozzles which can deploy droplets in the range of 15-40 microns is preferred. The ability to create a unique spray profile for a particular room configuration is a critical part of the invention. A fluid expelling region is located at a front of the housing 110 and has an opening through which atomized fluid is expelled.

A pump 320 is used to retrieve the sanitization fluid contained within reservoir and communicate the sanitization fluid to nozzle 315 for dispersion into the ambient environment. Preferably pump 320 can provide the sanitization fluid to the nozzle at a pressure of 35-130 psi.

To ensure efficient delivery of the pressurized fluid to the nozzle, rigid tubing 330 is preferably required.

A fan 400 is preferably utilized in assisting in the dispersion of the sanitization fluid. Fan 400 pulls air from the ambient environment through an opening in the housing. The fan preferably is variable for varying the CFM output for dispelling the fluid. The fan is preferably located axially behind the nozzle assembly in a manner that the fan directs a fluid flow of air linearly with the dispersion of the sanitization fluid from the nozzle to provide a current of air as a medium for transporting the sanitization fluid droplets as they exit the nozzle into the ambient air of the room for sanitization of the room and the associated objects. The volume and velocity of the current of air may be manipulated by a controller regulating the speed of the fan. Preferably the fan is continuously running which facilitates in expelling any residue from the nozzles once the sanitization process is completed which may drip and create a slipping hazard.

A power supply 500 is utilized for powering the components of the system. Power supply 500 may be a battery or an electrical power circuit which is plugged into a wall outlet.

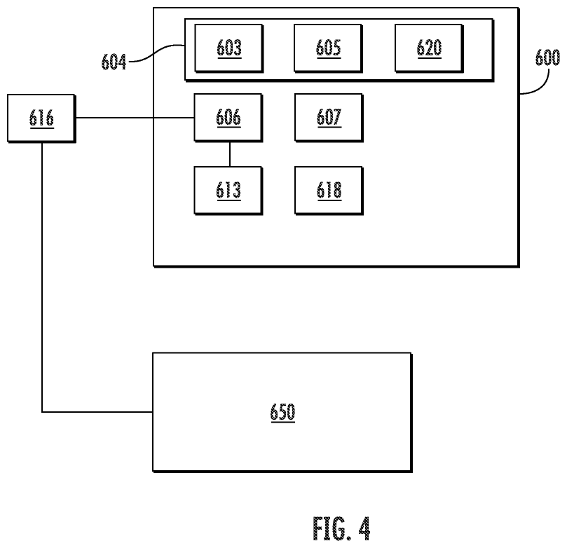

As shown in FIG. 4 central processing system 600 controls the operation of the components of the system. The central processing system may include a small computer system interface (SCSI) adapter that communicates with a hard disk 603 and/or tape storage drive 605 or any other similar component. I/O adapter 607, hard disk 603, and tape storage device 605 are collectively referred to herein as mass storage 604. Operating system 620 for execution on the processing system 600 may be stored in mass storage 604. A network adapter 606 interconnects bus 613 with an outside network 616 enabling system 600 to communicate with external management systems 650. Processor 618 executes instructions for operating the components of the system 100.

The system 100 is controlled and managed by central processing system 600. Each of the independent systems, i.e., the pump, nozzle assembly, fan and power system may be controlled by the central processing system 600 to facilitate in the dispensing of the sanitization fluid. A user via external management system 650 may monitor and control the operation of the system depending on desired circumstances. The central processing system may be operated remotely by a user utilizing various connectivity systems such as wi-fi; Bluetooth, or directly wired to a central control system 122 which may be operated by a central system at a multi-room establishment such as a hotel or a hospital for controlling the operation of multiple systems 100 which may be installed in individual locations within the multi-room establishment.

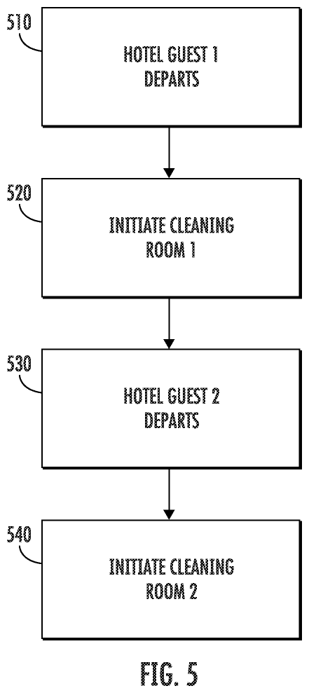

In this manner a network of sanitization systems may be integrated to facilitate the sanitization of the multi-room establishment. As shown in FIG. 5. For instance, at a hotel, at the check-in check-out desk, when a first guest departs at 510, the sanitization system 100 of the respective room is initiated to clean the corresponding room at 520. The system may automatically initiate operation of the sanitization system for the respective room merely upon receiving an instruction from the centralized administration system that the party has checked out, or alternatively, the clerk may initiate instructions for initiation of the sanitization process. By networking the sanitization system with alternative administration systems, the sanitization of the rooms may be easily coordinated and conducted. The respective disinfecting systems may be utilized in multiple room establishments such as hotels and hospitals. As shown in FIG. 5 when the second hotel guest departs at 530, the sanitization system 100 of the respective room is initiated to clean the corresponding room at 540.

The respective disinfecting systems can be networked to a central control center such as a front desk of the hotel. In such an environment the following configuration may be utilized. A disinfecting system for a first room having at least a first and second wall defining a first room dimension and a second room having at least a first and second wall defining a second room dimension. The system includes a first disinfecting unit carried within the first room comprising a housing and a reservoir containing a disinfecting fluid carried by the housing. A nozzle assembly carried by the housing including a nozzle and a nozzle controller for dispensing the disinfecting fluid into a room. A pump for communicating the disinfecting fluid from the reservoir to the nozzle assembly and a controller for controlling the dispensing of the disinfectant. A first dispensing profile for controlling the dispensing of the disinfectant. The first dispensing profile utilizing the dimensions of the first room as instructions for the central controller to identify the range for dispensing the disinfecting fluid from the first disinfecting unit. The central controller utilizing the first dispensing profile instructions for manipulating either the pump, fan or nozzle assembly to dispense a desired volume of the disinfectant fluid at a range as identified by the first dispensing profile to provide the disinfecting fluid to a first sanitization area as defined by the first dispensing profile and wherein the amount of sanitization fluid dispensed will remain in a fluid state within the room constituting a dwell time of at least between one minute and ten minutes.

Also, a second disinfecting unit carried within the second room having a housing and a reservoir containing a disinfecting fluid. A nozzle assembly including a nozzle and a nozzle controller for dispensing the disinfecting fluid into a room. A pump for communicating the disinfecting fluid from the reservoir to the nozzle assembly, the pump carried by the housing. A controller for controlling the dispensing of the disinfectant. The controller carried by the housing. A second dispensing profile for controlling the dispensing of the disinfectant. The second dispensing profile utilizing the dimensions of the second room as instructions for the central controller to identify the range for dispensing the disinfecting fluid from the second disinfecting unit. The central controller utilizing the second dispensing profile instructions for manipulating either the pump, fan or nozzle assembly to dispense a desired volume of the disinfectant fluid at a range as identified by the second dispensing profile to provide the disinfecting fluid to a second sanitization area as defined by the second dispensing profile. Wherein the amount of sanitization fluid dispensed will remain in a fluid state within the room constituting a dwell time of at least between one minute and ten minutes; and the first sanitization area being within the first room and the second sanitization area being within the second room.

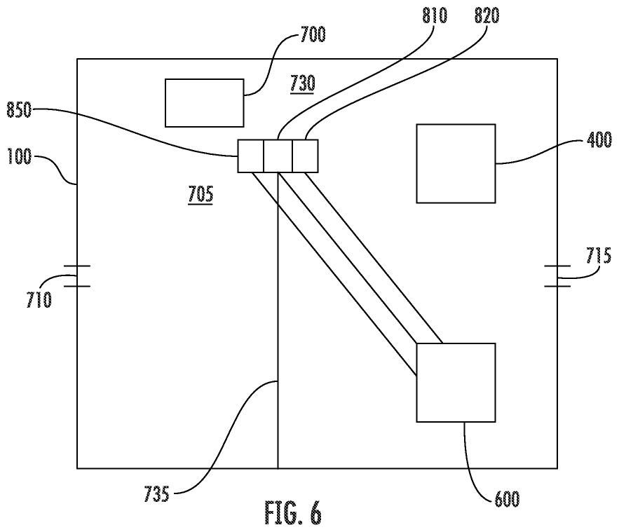

In an additional embodiment shown in FIG. 6, the system 100 may include a UV light system 610 for sterilizing the ambient air. The UV light system in combination with the sterilization spray system provides both continuous sterilization of the ambient air and a hard cleaning of the walls/ceilings and structures in the room. As shown in FIG. 6 sanitization system 100 includes a UV light system 700 within housing interior creating a UV treatment area 705. In the preferred embodiment, an air inlet 710 is formed within housing permitting ambient air to be drawn into the interior of the housing by the fan 400. An air outlet 715 is present within housing to permit the ambient air to exit the interior. An airflow pathway 730 created utilizing baffles 735 to direct the airflow from air inlet 710 to air outlet 715 and pass through UV treatment area 705. The fan is preferably constantly running in order to continuously draw air from the ambient environment through the UV treatment area 605.

By continuously drawing air from the ambient environment, the system has the ability to identify and collect sensed information from the ambient environment in order to modify or direct the operation of the sanitization system. In the preferred embodiment the system includes a humidity sensor 850, a temperature sensor 810 and a contamination sensor 820. Humidity sensor 850 senses the humidity within the ambient environment and provides the humidity reading to the central processing system 600. Temperature sensor 810 senses the temperature of the ambient environment and provides the temperature reading to the central processing system 600. The central processing unit may calculate the relative humidity based upon the sensed humidity and temperature. In the preferred embodiment, a contamination sensor 820 of the type known as a MINIMA manufactured by Applied Particle Technology may monitor the ambient environment to determine the presence of certain germs such as COVID-19. Each sensor is located with airflow pathway 730 in order to receive the ambient air and sense the respective condition of the air with the respective sensor.

With the information of the humidity, temperature and presence of contaminants, the central processing system may operate the various systems of the sanitization system to provide a varying sanitization effort. For example, by measuring the humidity at the time that the sanitization process is initiated and during the sanitization process, if the humidity increases to a level above a desired level, this may indicate that the air is oversaturated with sanitization fluid and central processing system may suspend the sanitization process until the humidity is lower. Or alternatively, if a certain dwell time such as ten minutes is required, the central processing system may note the humidity level at the time the sanitization process is initiated, and also the levels during the sanitization process, if the humidity level drops to the original level prior to the ten minute dwell time occurring, as measured by the system utilizing a clock for measuring the time that the sanitization process is in effect, then the central processing system may initiate a second sanitization process.

Additionally, if a contaminant is identified, the central processing unit may include a dwell table profile for respective contaminants as identified in Attachment A and control the operation of the system to ensure that the respective required dwell time is accomplished.

A critical feature of the invention is providing a sanitization system which effectively sanitizes a room and the associated objects within the room by ensuring that sufficient sanitization fluid is provided to ensure a certain dwell time for interacting with possible contaminants and killing them while also not over saturating the room which results in the room being unusable for an extended period of time due to the hazard created by a slippery environment. The system is intended to sterilize a room with certain size and ambient characteristics in a manner which enables the room to be effectively sanitized without oversaturating the environment which produces a hazardous situation and increases the time which the particular room would not be suitable for occupation or utilization. By considering the characteristics of the room, an effective and efficient sanitization profile may be established.

The inventors have discovered that utilizing the charged Hypochlorous Acid that a dispersion pattern resulting in approximately 14,300 droplets per inch at twenty feet away from the sanitizing system 100 was achieved which allowed for full coverage of the respective surface areas within seconds and which provided for a dwell time of at least ten minutes sufficient to kill 99.9 percent of contaminants. By utilizing the 14,300 dpi requirement at twenty feet, this volume assured full coverage of the sprayed area but of a quantity which fully evaporated at approximately ten minutes enabling the area to be utilized. This finding enables a dispensing profile to be created for ranges which include twenty feet and less. For a less distance, the system is configured to taper back the operation to provide approximately only 14,300 at the lesser area. This dynamic dispensing profile is critical to the invention as it ensures maximum coverage with the minimum quantity of dispensing fluid thus enabling the room to be reused as quickly as possible.

A unique aspect of the invention is the evaluation of the room parameters in order to direct the sanitization system to optimally direct sanitization fluid into the ambient environment. A primary feature of the invention is the creation of a sanitization profile which is created based upon the room's configuration. The room's configuration is utilized to create a sanitization profile 900 which the processor utilizes to control the operation of the sanitization system to control the dispersion of the quantity of sanitization fluid as directed by the sanitization profile into the room in a manner to effectively sanitize the room and its respective contained features while preventing oversaturation of the ambient environment.

For example, the system may be created with components which can produce a stream of sanitization fluid which extends twenty feet from the nozzle tip and has a width of four feet. However, the system may be positioned along a wall which is only ten feet from an abutting neighbor wall. Utilizing the full twenty-five foot stream would be excessive resulting in overabundance of sanitizing fluid which would pool and collect on the wall and floor resulting in an extended time for the fluid to evaporate. This extended time results in the room becoming unavailable for occupation for an extended period of time. The invention solves this problem by controlling the dispersion of sanitization fluid only to the extend needed to perform the sanitization process.

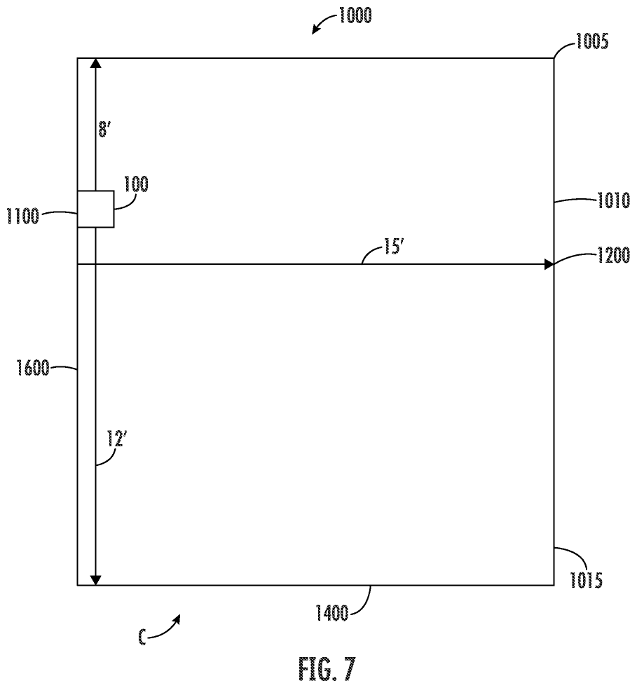

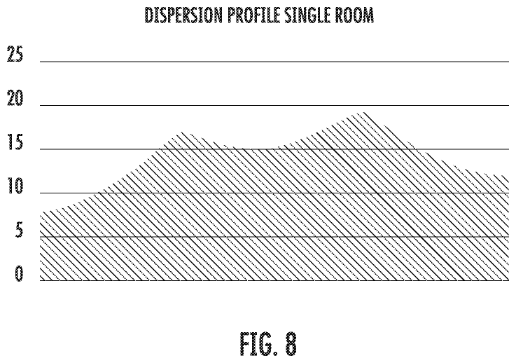

FIG. 7 illustrates the creation of the dispersion profile 900. Room C has a predefined configuration of a depth D, width E and height F with walls 1000, 1200, 1400, 1600 and ceiling not shown. Sanitization system 100 is positioned along wall 16 beneath ceiling 2000. Preferably sanitization system 100 is positioned one foot below ceiling at position 1100. The distances from position 1100 to wall 1200 is determined. Preferably a LiDAR, light detection and ranging sensor is utilized for determining the relative distances from the mounted sanitization system 100 at position 1100 and the respective walls 1200, 1400 and 1600. Alternatively, the relative distances could be manually measured and inputted into the memory of the device for utilization by the controller. An example of a sanitization profile 900 may be illustrated as follows. It may be determined that wall 1000 is eight feet away from sanitization box 100 at position 1100. Wall 100 may be fifteen feet in length away from the box, e.g., the width of the room, such that the closest position of wall 1000 to sanitization box 1000 is eight feet, but the farthest position which would be the hypotenuse is 17 feet at corner 1005. With respect to opposite wall 1200, which in this example is fifteen feet opposite from wall 1600, and has a length twenty feet, the distance to corner 1005 is seventeen feet, this distance decreases as to fifteen feet directly across from the sanitization box 100 at position 1010 and then increases to a maximum distance of nineteen feet, the hypotenuse as measured to the opposite corner 1015. The central processing unit may manipulate the operation of the fan, pump, or delivery time to manipulate the volume of dispensed sanitization fluid to ensure that the appropriate amount is delivered to ensure a minimum dwell time. FIG. 8 reflects the dispersion profile 900 for the room shown in FIG. 7. The corresponding data which comprises the dispersion profile is shown in Table A. In operation, the system sprays a stream for five seconds down along wall 1600. The stream is wide enough to contact wall 1600 and the five seconds is preferably sufficient time to provide a layer of disinfectant to cover wall 1600. Likewise, when the dispersion profile is completed and the remainder of wall 1600 requires coverage, the duration of the spray is also five seconds. In an experiment, the entire room received sufficient coverage in under fifty eight second

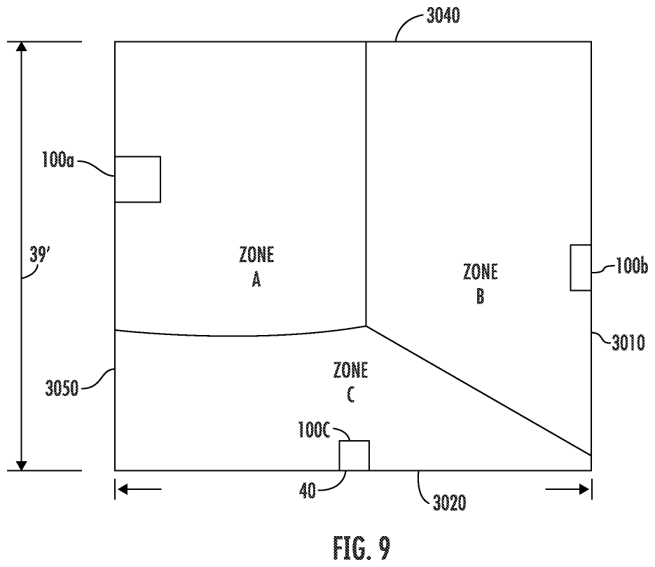

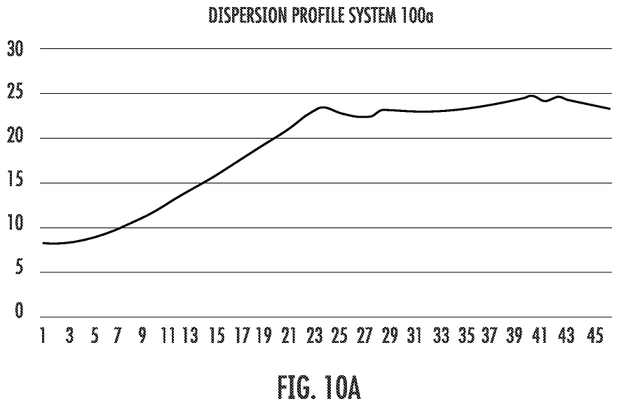

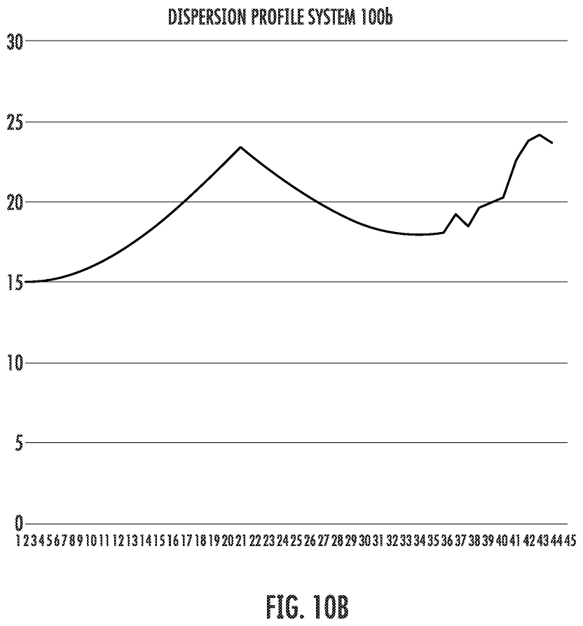

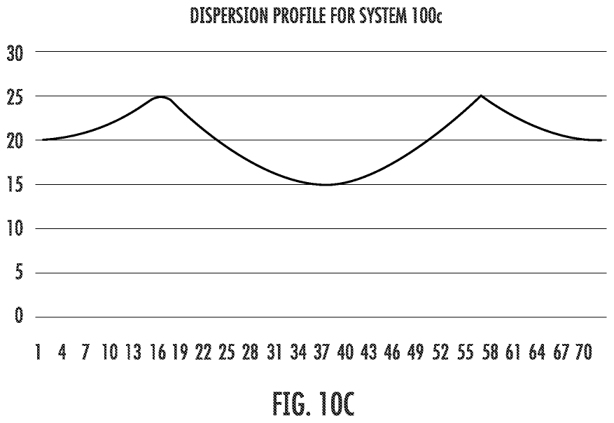

FIG. 9. Illustrates an additional room configuration with a system of multiple sanitization systems 100a, 100b, and 100c. In this configuration the room is rectilinear and is forty feet wide having a length of thirty nine feet. System 100a is positioned along wall 3000 eight feet away from wall 3040, System 100b is positioned along wall 3010 fifteen feet away from wall 3040 and System C is positioned along wall 3020 and the halfway twenty foot mark. The respective dispersion profiles for each system is illustrated in FIGS. 10A. 10B, and 10C respectively. By utilizing multiple sanitization systems with different sanitization dispersion profiles, the entire room may be sanitized in a dispersion cycle which is less than a minute and a half. Table B consists of the data utilized for creating the respective dispersion profiles for the respective systems.

As shown, the dispersion profiles illustrate that the flow of sanitization fluid varies throughout the sanitation process as the nozzle assembly rotates the nozzle from a left to right spray pattern. The volume of fluid is varied depending on the distance which the stream must carry in order to reach a wall, this distance varies depending on the size of the room and the location of the sanitization system. The establishment of the dispersion profile is the critical feature of the invention as it facilitates in only dispersing the required amount to land on an extended target. By electrostatically charging the sanitization fluid, the fluid carries through the air due to the molecular attraction of the different polarities and also the attraction of the opposite charged wall. By electrostatically charging the sanitization fluid, less power is required by the system as dispersion is facilitated by the ambient air itself, and less sanitization fluid is required to contact a surface as the sanitization fluid will creep along the oppositely charged surfaces to cover the entire surface.

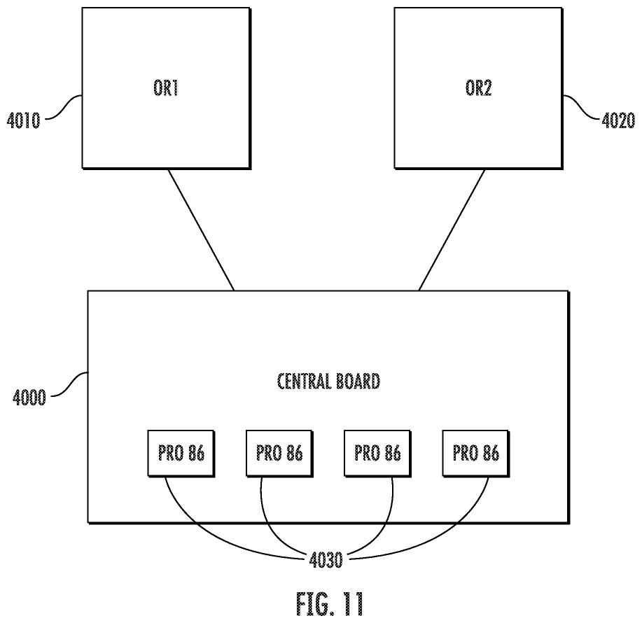

As shown in FIG. 11, a critical implementation of the sanitization system in the hospital setting either for patient care rooms or operating rooms. A central control network 4000 may be utilized for controlling certain aspects of an operating room 4010 and 4020 such as temperature, lighting and the like. A sanitization system may be integrated with such a control network and when an operation is completed, the sanitization system may be initiated by the central control administrator. For the operating room, the sanitization system may have various operating profiles depending on the contaminant perceived to be present in the operating room. The perceived contaminants may be classified as followed: touching, airborne, spray, or by type such as MSRA, TB etc. These contaminants may require different dwell times in order to accomplish certain kill rate goals, such as ninety-nine percent kill. These contaminants may include those which are identified as contact precautions, such as a MSRA, a droplet precaution, or an airborne precaution. Also with Sars another level of precautions is required. A demonstrative list of certain bacteria, fungi, mold and viruses which are intended to be sanitized are shown in Attachment C. These dwell times may range from one minute to ten minutes. Accordingly, the sanitization system will include various dispensing profiles 4030 such as Profile 1 for a MSRA, Profile 2 for TB, Profile 3 for an airborne and Profile 4 for Hepatitis or the profiles will vary for contact precautions, droplet precautions airborne precautions and Sars precautions. These profiles vary depending on the circumstances of the contaminant in the room and will ensure that the dispensed sanitation fluid will provide for an effective dwell time ranging from one minute to ten minutes depending on the perceived contaminate. The dispensing profiles will correspond to the type of contaminant such as touch, airborne etc. In operation, upon initiation by the end user, the end user will input the required dispensing profile such as touch, airborne, etc, into the system and the central processing system will utilize the dispensing profile for the room which considers the dimension of the room and the respective dwell time necessary.

As it may be seen, a more advantageous sanitization system may be had. In the preferred embodiment the mechanical operations of the sanitization device include a bladder that is a maximum of two liters with a fill-to line. This is a self-contained reservoir attached to a transport line made of a quarter inch rigid tubing, approximately five inches to the pump. The pump is a twelve volt unit with a direct current maintaining a three liter per minute flow with a maximum pressure of eighty psi. A dispensing line communicates with the pump for dispensing a sanitization fluid to a nozzle assembly. The dispensing line is approximately two feet in length of three eighths inch rigid tubing and communicates with a nozzle assembly which may oscillate a one hundred and seventy-five degrees from a position pointing to the left to a position pointing to the right. The nozzle assembly includes adjustability in the vertical direction above and below a central line. The nozzles may range from fifty to eighty micron tips. A fan provides sufficient cfm to carry eighty psi of sanitization fluid to a maximum range of twenty-five feet. A sanitization dispensing profile is established based upon the size of the room and location of the sanitization device within the room. The sanitization dispensing profile is utilized by a central processing unit for controlling the operation of either the pump pressure, the operational time of the pump, the fan's operation for manipulating the output cfm, either individually or in combination to facilitate the required fluid dispensing for the desired sanitization stream. The stream may range from one foot to twenty-five feet.

In certain room configurations, a plurality of sanitization devices may be required in order to provide sufficient sanitization fluid to cover the required area. In these arrangements, the respective sanitization devices will have their own unique sanitization dispensing profiles.

1: A disinfecting system for a room having at least a first and second wall defining a room dimension, comprising:
a housing;
a reservoir containing a disinfecting fluid carried by said housing;
a nozzle assembly including a nozzle and a nozzle controller for dispensing said disinfecting fluid into a room, said nozzle assembly carried by said housing;
a pump for communicating said disinfecting fluid from said reservoir to said nozzle assembly, said pump carried by said housing;
a controller for controlling the dispensing of said disinfectant, said controller carried by said housing;
a dispensing profile for controlling the dispensing of said disinfectant;
said dispensing profile utilizing the dimensions of the room as instructions for said central controller to identify the range for dispensing said disinfecting fluid, said central controller utilizing said dispensing profile instructions for manipulating either said pump, fan or nozzle assembly to dispense a desired volume of said disinfectant fluid at a range as identified by said dispensing profile;
wherein the amount of sanitization fluid dispensed will remain in a fluid state within the room constituting a dwell time of at least between one minute and ten minutes.

2. The disinfectant system of claim 1 wherein said disinfectant is Hypochlorous Acid

3: The disinfectant system of claim 1 including a fan, wherein said fan is a variable fan having variable fan speeds and said controller manipulates the speed of the fan to increase the cfm of the fan in response to the dispersion profile.

4. The disinfectant system of claim 1 including an electrostatically charging element for electrostatically charging said disinfectant upon dispensing by said system.

5. The disinfectant system of claim 4 wherein the electrostatically charging element is positioned in axial alignment with a nozzle for electrostatically charging a disinfectant as the disinfectant is being dispensed by said nozzle.

6. The disinfectant system of claim 1 wherein said dispensing profile includes varying profiles depending on the type of containment intended to be disinfected and wherein said varying profile includes profiles for contaminants identified a contact precautions, droplet precautions, or airborne precautions.

7. The disinfectant system of claim 1 including an ultra-violet lighting system carried within the interior of said housing for providing sanitization treatment to the ambient environment wherein airflow is established by the fan drawing air into the housing from an inlet and said housing including an outlet for dispensing ultra-violet treated air.

8. The disinfectant system of claim 7 including a humidity sensor located within an airflow path between said inlet and outlet for measuring the humidity of the ambient air, said measurement of said humidity transferred to said controller for controlling the dispensing of said disinfectant.

9: A disinfecting system for a room having at least a first and second wall defining a room dimension, comprising:
A first disinfecting unit comprising:
a housing;
a reservoir containing a disinfecting fluid carried by said housing;
a nozzle assembly including a nozzle and a nozzle controller for dispensing said disinfecting fluid into a room, said nozzle assembly carried by said housing;
a pump for communicating said disinfecting fluid from said reservoir to said nozzle assembly, said pump carried by said housing;
a controller for controlling the dispensing of said disinfectant, said controller carried by said housing;
a first dispensing profile for controlling the dispensing of said disinfectant;
said first dispensing profile utilizing the dimensions of the room as instructions for said central controller to identify the range for dispensing said disinfecting fluid from said first disinfecting unit, said central controller utilizing said first dispensing profile instructions for manipulating either said pump, fan or nozzle assembly to dispense a desired volume of said disinfectant fluid at a range as identified by said first dispensing profile to provide said disinfecting fluid to a first sanitization area as defined by said first dispensing profile;
wherein the amount of sanitization fluid dispensed will remain in a fluid state within the room constituting a dwell time of at least between one minute and ten minutes; and
a second disinfecting unit comprising:
a housing;
a reservoir containing a disinfecting fluid carried by said housing;
a nozzle assembly including a nozzle and a nozzle controller for dispensing said disinfecting fluid into a room, said nozzle assembly carried by said housing;
a pump for communicating said disinfecting fluid from said reservoir to said nozzle assembly, said pump carried by said housing;
a controller for controlling the dispensing of said disinfectant, said controller carried by said housing;
a second dispensing profile for controlling the dispensing of said disinfectant;
said second dispensing profile utilizing the dimensions of the room as instructions for said central controller to identify the range for dispensing said disinfecting fluid from said second disinfecting unit, said central controller utilizing said second dispensing profile instructions for manipulating either said pump, fan or nozzle assembly to dispense a desired volume of said disinfectant fluid at a range as identified by said second dispensing profile to provide said disinfecting fluid to a second sanitization area as defined by said second dispensing profile;
wherein the amount of sanitization fluid dispensed will remain in a fluid state within the room constituting a dwell time of at least between one minute and ten minutes; and
said first sanitization area is different than said second sanitization area.

10. The sanitization system of claim 9 wherein said first and second sanitization areas encompass the entire area of the room.

11. The sanitization system of claim 9 including a third disinfecting unit comprising:
a housing;
a reservoir containing a disinfecting fluid carried by said housing;
a nozzle assembly including a nozzle and a nozzle controller for dispensing said disinfecting fluid into a room, said nozzle assembly carried by said housing;
a pump for communicating said disinfecting fluid from said reservoir to said nozzle assembly, said pump carried by said housing;
a controller for controlling the dispensing of said disinfectant, said controller carried by said housing;
a third dispensing profile for controlling the dispensing of said disinfectant;
said third dispensing profile utilizing the dimensions of the room as instructions for said central controller to identify the range for dispensing said disinfecting fluid from said third disinfecting unit, said central controller utilizing said third dispensing profile instructions for manipulating either said pump, fan or nozzle assembly to dispense a desired volume of said disinfectant fluid at a range as identified by said third dispensing profile to provide said disinfecting fluid to a third sanitization area as defined by said third dispensing profile;
wherein the amount of sanitization fluid dispensed will remain in a fluid state within the room constituting a dwell time of at least between one minute and ten minutes; and
said third sanitization area is different than said first or said second sanitization area.

12. A disinfecting system for a first room having at least a first and second wall defining a first room dimension and a second room having at least a first and second wall defining a second room dimension, comprising:
a first disinfecting unit carried within said first room comprising:
a housing;
a reservoir containing a disinfecting fluid carried by said housing;
a nozzle assembly including a nozzle and a nozzle controller for dispensing said disinfecting fluid into a room, said nozzle assembly carried by said housing;
a pump for communicating said disinfecting fluid from said reservoir to said nozzle assembly, said pump carried by said housing;
a controller for controlling the dispensing of said disinfectant, said controller carried by said housing;
a first dispensing profile for controlling the dispensing of said disinfectant;
said first dispensing profile utilizing the dimensions of the first room as instructions for said central controller to identify the range for dispensing said disinfecting fluid from said first disinfecting unit, said central controller utilizing said first dispensing profile instructions for manipulating either said pump, fan or nozzle assembly to dispense a desired volume of said disinfectant fluid at a range as identified by said first dispensing profile to provide said disinfecting fluid to a first sanitization area as defined by said first dispensing profile;
wherein the amount of sanitization fluid dispensed will remain in a fluid state within the room constituting a dwell time of at least between one minute and ten minutes; and
a second disinfecting unit carried within said second room comprising:
a housing;
a reservoir containing a disinfecting fluid carried by said housing;
a nozzle assembly including a nozzle and a nozzle controller for dispensing said disinfecting fluid into a room, said nozzle assembly carried by said housing;
a pump for communicating said disinfecting fluid from said reservoir to said nozzle assembly, said pump carried by said housing;
a controller for controlling the dispensing of said disinfectant, said controller carried by said housing;
a second dispensing profile for controlling the dispensing of said disinfectant;
said second dispensing profile utilizing the dimensions of the second room as instructions for said central controller to identify the range for dispensing said disinfecting fluid from said second disinfecting unit, said central controller utilizing said second dispensing profile instructions for manipulating either said pump, fan or nozzle assembly to dispense a desired volume of said disinfectant fluid at a range as identified by said second dispensing profile to provide said disinfecting fluid to a second sanitization area as defined by said second dispensing profile;
wherein the amount of sanitization fluid dispensed will remain in a fluid state within the room constituting a dwell time of at least between one minute and ten minutes; and
said first sanitization area being within said first room and said second sanitization area being within said second room.

13. The system of claim 12 wherein said first and said second disinfecting units are controlled by a centralized operating system. desk.

14. The system of claim 12 wherein the centralized operating system is a hotel front

15. The system of claim 12 wherein the centralized operating system is a hospital operating room control system.

16. The system of claim 15 wherein the centralized operating system is a hospital Command and Control system which controls the dismissal of hospital patients.

17. In an operating room, a disinfecting system comprising:
a housing having an interior;
a reservoir containing a disinfecting fluid carried within said interior;
a nozzle assembly including a nozzle and a nozzle controller for dispensing said disinfecting fluid into a room;
a pump for communicating said disinfecting fluid from said reservoir to said nozzle assembly;
an electrostatic charging assembly for electrostatically charging said disinfectant exiting said nozzle;
a dispensing profile for controlling the dispensing of said disinfectant;
said dispensing profile utilizing the dimensions of the room as instructions for said central controller, said central controller utilizing said dispensing profile instructions for manipulating either said pump, fan or nozzle assembly to dispense a desired volume of said disinfectant fluid at a range as identified by said dispensing profile;
a plurality of contaminant dispensing profiles which includes a first contaminant profile for instructing the controller to dispense a desired amount of disinfecting fluid corresponding to a first contaminant having a dwell time, a second contaminant profile for instructing the controller to dispense a desired amount of disinfecting fluid corresponding to a second contaminant having a required dwell time of at least ten minutes;
a central controller for processing information relating to said dispensing profile and said contaminant dispensing profile for manipulating the operation of either said pump, fan or nozzle assembly for controlling the dispersion of said disinfecting fluid into said room;
wherein said dwell time is achieved via said central controller controlling the dispersion of said disinfectant fluid.

18. The disinfectant system of claim 17 including an electrostatically charging element for electrostatically charging said disinfectant upon dispensing by said system.

19. The disinfectant system of claim 18 wherein the electrostatically charging element is positioned in axial alignment with a nozzle for electrostatically charging a disinfectant as the disinfectant is being dispensed by said nozzle.

20. The disinfectant system of claim 17 wherein said dispensing profile includes varying profiles depending on the type of containment intended to be disinfected and wherein said varying profile includes profiles for contaminants identified as contact precautions, droplet precautions, or airborne precautions.
